(19) United States
(12) Patent Application Publication
(10) Pub. No.: US 2010/0217018 A1
(43) Pub. Date: Aug. 26, 2010

(54) Method for the Production of Substituted and Unsubstituted Cyclohexanone Monoketals

(75) Inventors: Wahed Ahmed Moradi, Monheim (DE); Matthias Decker, Koln (DE); Gunter Karig, Hofheim am Taunus (DE); Thomas Himmler, Odenthal (DE); Norbert Lui, Odenthal (DE); Alexander Straub, Wuppertal (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer CropScience AG, Monheim (DE)

(21) Appl. No.: 12/738,316

(22) PCT Filed: Oct. 16, 2008
(86) PCT No.: PCT/EP08/08766

§ 371 Date: Apr. 16, 2010

(30) Foreign Application Priority Data
Oct. 29, 2007 (EP) 07119476.5

(51) Int. Cl. C07D 319/04; C07D 317/72; C07C 45/00

(52) U.S. Cl. 549/333; 549/341; 568/309

(57) ABSTRACT

The invention relates to a novel process for preparing and isolating known substituted and unsubstituted 1,4-cyclohexanone monoketals.

The present invention relates to a novel process for preparing and isolating known substituted and unsubstituted 1,4-cyclohexanone monoketals.

Substituted and unsubstituted cyclohexanone monoketals are important starting materials for synthesizing active ingredients for crop protection and drugs, such as, for example, the therapeutic agent for migraine frovatriptan.

A number of different processes are known in the literature for preparing cyclohexanone monoketals:

for example, US2004/0230063 describes the sulphuric acid-catalysed monoketalization of 1,4-cyclohexanedione with one equivalent of neopentyl glycol to give a mixture of dione, monoketal and bisketal which are difficult to separate. As a result, the working up is very elaborate, which is a great disadvantage for an economic process on the industrial scale. Nor does the use of other diols make selective monoketalization and simplification of the working up possible.

J. Org. Chem. 1983 48, 129-131 describes the monoketalization of 1,4-cyclohexanedione with 1,4-butanediol, again resulting in a mixture of mono- and bisketal. In addition, the isolated yield after an elaborate working up is only 59% of theory and, moreover, the reaction is difficult to carry out on the industrial scale. It should further be mentioned that the starting compound 1,4-cyclohexanedione is a costly item.

A further method for preparing monoketals is described in Bull. Soc. Chim. Fr. 1983, 3-4, 87-88, in a reaction of 1,4,9,12-tetraoxadispiro[4.2.4.2]tetradecane with 1,4-cyclohexanedione. However, the yield for this process is reported to be 74% and requires a very elaborate working up which is costly on the industrial scale.

There are also descriptions in the literature of controlled monodeketalizations of bisketals. For example, monodeketalization without solvent using iron(III) chloride on silica gel is reported in Synthesis 1987, 37-40. It is noteworthy in this connection that the bisketal 1,4,9,12-tetraoxadispiro[4.2.4.2]tetradecane is a solid. This process therefore also has the disadvantages described above, and industrial implementation of such a process is therefore scarcely practicable.

In view of the disadvantages and problems described above, there is a pressing need for a simplified process which can be carried out industrially and economically for the selective preparation of substituted and unsubstituted 1,4-cyclohexanone monoketals on the industrial scale.

It has been found that compounds of the formula (I)

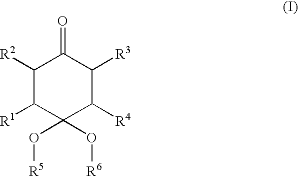

in which
R¹, R², R³, R⁴ independently of one another are hydrogen or are in each case optionally mono- or polysubstituted C₁-C₄-alkyl or cyclopropyl,
R⁵ and R⁶ independently of one another are C₁-C₈-alkyl or are cycloalkyl, or
R⁵ and R⁶ together are —CH₂—, —CH₂CH₂—, —CH₂CH₂CH₂—, —CH₂CH₂CH₂CH₂—, —CHCH₃CH₂CHCH₃CH₂—, —CH₂C(CH₃)₂CH₂, CH₂OCH₂—, —CH₂OCH₂CH₂— or —CH₂CH₂OCH₂—,
are obtained by hydrogenating compounds of the formula (II)

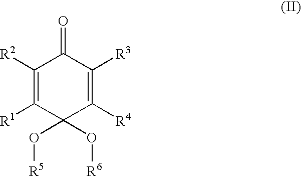

in which
R¹, R², R³, R⁴, R⁵, R⁶ have the meanings indicated above,
in the presence of a suitable metal catalyst, of a suitable additive and where appropriate of a solvent.

In the general formulae (I) and (H), the substituents

R¹, R², R³, R⁴ independently of one another preferably are hydrogen or are methyl, ethyl, i-propyl, t-butyl or cyclopropyl,
R⁵ and R⁶ independently of one another preferably are methyl, ethyl, i-propyl, t-butyl or are cyclopropyl, cyclobutyl, cyclopentyl, or cyclohexyl or
R⁵ and R⁶ together preferably are —CH₂—, —CH₂CH₂—, —CH₂CH₂CH₂—, —CH₂CH₂CH₂CH₂—, —CHCH₃CH₂CHCH₃CH₂— or —CH₂C(CH₃)₂CH₂—.

In the general formulae (I) and (H), the substituents

R¹, R², R³, R⁴ independently of one another particularly preferably are hydrogen, methyl or ethyl,
R⁵ and R⁶ independently of one another particularly preferably are methyl, ethyl, i-propyl or t-butyl or R⁵ and R⁶ together particularly preferably are —CH₂—, —CH₂CH₂—, —CH₂CH₂CH₂—, —CHCH₃CH₂CHCH₃CH₂— or —CH₂C(CH₃)₂CH₂—.

In the general formulae (I) and (II), the substituents

R¹, R², R³, R⁴ independently of one another very particularly preferably are hydrogen or methyl, (emphasized for hydrogen),
R⁵ and R⁶ independently of one another very particularly preferably are methyl, or
R⁵ and R⁶ together very particularly preferably are —CH₂CH₂— or —CH₂CH₂CH₂—.

The definitions of radicals and explanations specified above in general or specified in preferred ranges can be combined with one another as desired, i.e. also between the respective ranges and preferred ranges.

Particular preference is given to the compound of the formula (I-1)

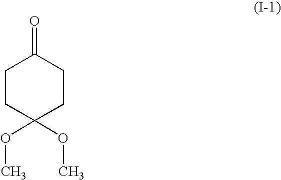

which is obtained by hydrogenating the compound of the formula (II-1)

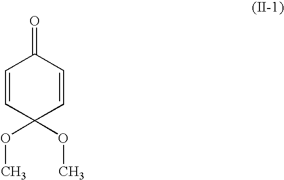

in the presence of a suitable metal catalyst, of a suitable additive and where appropriate of a solvent.

Particular preference is further given to the compound of the formula (I-2)

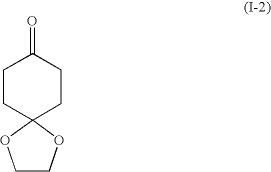

which is obtained by hydrogenating the compound of the formula (II-2)

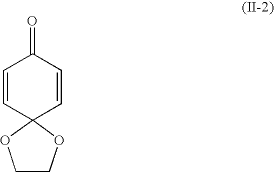

in the presence of a suitable metal catalyst, of a suitable additive and where appropriate of a solvent.

Particular preference is further given to the compound of the formula (I-3)

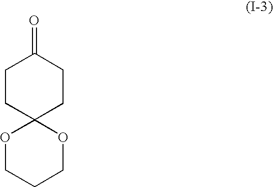

which is obtained by hydrogenating the compound of the formula (II-3)

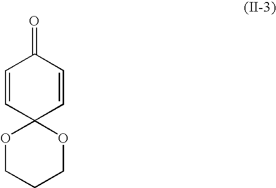

in the presence of a suitable metal catalyst, of a suitable additive and where appropriate of a solvent.

The compounds of the formulae (I) and (II) are disclosed in the literature.

The compounds of the formula (II) are obtained by monodeketalization of bisketals by known methods of ketal hydrolysis in the presence of a catalytic amount of an organic or inorganic acid or in a solvent mixture (Protective Groups in Organic Synthesis, T. Greene and P. Wuts, Wiley-Interscience).

Hydrogenation of compounds of the formula (II) has to date been confined in the literature to sterically very demanding, stable and comparatively very costly cyclic ketals. A process of this type is described by March et. al. in Tetrahedron Asymmetry 2003, 14, 2021-2032. In this case, 2,3-diphenylspiro[4.5]decan-8-one is obtained by palladium-catalyzed hydrogenation of 2,3-diphenylspiro[4.5]deca-6,9-dien-8-one in toluene.

In the state of the art, hydrogenation of compounds of the formula (II) with very costly cyclic ketals may result in subsidiary components, for example (A) and (B) in Scheme 1.

It has now surprisingly been found that even very reasonably priced and easily available and thus industrially interesting compounds of the formula (II) such as, for example, 4,4-dimethoxycyclohexa-2,5-dien-1-one of the formula (II-1) or 1,4-dioxaspiro[4.5]deca-6,9-dien-8-one (II-2) or 1,5-dioxaspiro[5.5]undeca-7,10-dien-9-one (II-3) can be hydrogenated under very mild reaction conditions undiluted or in a solvent.

It has surprisingly likewise been found that the formation of the subsidiary component of the formula (B) as shown in Scheme 1 can be avoided or reduced to traces by addition of a base and in suitable solvents such as, for example, toluene, methyltetrahydrofuran or ethyl acetate. Possible subsidiary components of the formula (B) can be removed after the hydrogenation in the subsequent working up with sodium hydroxide solution.

The formation of subsidiary component (A) is preferred at high pressures, in polar solvents and longer reaction times and can thus be reduced or even completely avoided by adjusting the reaction conditions.

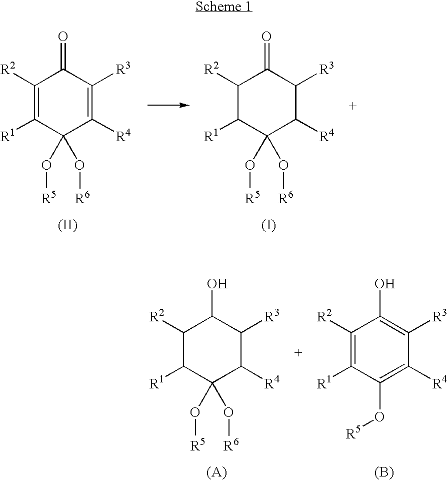

It is thus possible by the process of the invention to avoid or greatly reduce the formation of subsidiary components. The compounds of the formula (I) can therefore be prepared in very good yields and selectivities.

The compounds of the formula (II) are hydrogenated under atmospheric pressure or superatmospheric pressure with hydrogen in the presence of an active metal catalyst, of a nonpolar solvent and of an additive such as, for example, of a base.

Possible and suitable catalytically active metal compounds are all catalysts familiar to the skilled person for this purpose. These are preferably compounds of the metals of transition group 8 to 10 of the Periodic Table. Palladium metal catalysts are preferred. It is possible to employ as palladium catalysts or precatalysts any palladium(II) compounds, palladium(0) compounds and palladium on any usual inorganic support material such as, for example, alumina, silica, zirconia, titania or carbon, particularly preferably palladium on activated carbon. It has emerged that an amount of from 0.0001 to 5 mol % of the catalytically active metal compound (calculated as the metal), preferably 0.001 to 3 mol % based on the precursor are sufficient for the present process.

Suitable bases which can be used are all inorganic and organic bases considered by the skilled person for this purpose, such as, for example, alkali metal acetates, alkali metal and alkaline earth metal carbonates or bicarbonates, borax or organic bases such as trialkylamines, for example 1,5-diazabicyclo[5.4.0]undec-7-ene, triethylamine, tri-n-butylamine or diisopropylethylamine or a mixture thereof. The use of triethylamine, tri-n-butylamine or diisopropylethylamine is preferred. The stoichiometry of the base employed for the present process may vary within wide ranges and is generally subject to no special restriction. Thus, the molar ratio of the base to the precursor can be for example 0.001 to 5, particularly 0.01 to 2, specifically 0.01 to 0.1. The use of larger amounts of base is possible in principle but has no advantages.

Solvents which can be employed are water and all organic compounds familiar to the skilled person. Examples thereof are dioxane, tetrahydrofuran, methyltetrahydrofuran, ethylene glycol dimethyl ether, 1,2-dimethoxyethane, ethyl acetate, acetone, tert-butyl methyl ketone, xylene, toluene, alcohols such as, for example, methanol or mixtures thereof. The solvents can be employed in pure form or containing product or saturated with product. Preferred solvents are toluene, ethyl acetate and methyltetrahydrofuran.

The hydrogenation is preferably carried out at a temperature of 0-150° C., particularly preferably at 20 to 100° C., with the hydrogen pressure normally being from 1 to 150 bar, preferably from 5 to 100 bar. A reaction time of from 0.01 to 100 hours is usually sufficient.

The following exemplary embodiments explain the invention. The invention is not restricted to the examples.

### PREPARATION EXAMPLES

### Synthesis of 3,3,6,6-tetramethoxycyclohexa-1,4-dione [15791-03-4]

104 g (0.753 mol) of 1,4-dimethoxybenzene and 8 g (0.143 mol) of potassium hydroxide are dissolved in 500 ml of methanol. Then a platinized titanium anode and a nickel cathode is immersed in the reaction solution. The reaction mixture is electrolysed at room temperature in an unseparated flat flow cell at 0.65 A and a cell voltage of 22 V using a TG 96 laboratory potentiostat. The reaction is followed by gas chromatography. After the reaction is complete, the solvent is substantially removed under reduced pressure, and the residue is taken up in tert-butyl methyl ether and washed with water. 144 g (0.698 mol, 97% purity, 92.8% yield) of 3,3,6,6-tetramethoxycyclohexa-1,4-dione are obtained.

### Synthesis of 4,4-dimethoxycyclohexa-2,5-dien-1-one (II-1) [935-50-2]

120 g (0.6 mol) of 3,3,6,6-tetramethoxycyclohexa-1,4-dione are stirred in a mixture of 480 ml of tetrahydrofuran, 60 ml of water and 6 ml of acetic acid at 70° C. for 6 hours. After the monohydrolysis is complete, the tetrahydrofuran is stripped off in vacuo. The aqueous residue is mixed with 100 ml of NaHCO₃ solution and extracted 2× with 300 ml of tert-butyl methyl ether. The combined organic extracts are dried with Na₂SO₄, filtered through basic alumina and concentrated in a rotary evaporator. 88.4 g (0.562 mol, 98% purity, 95.7% yield) of 4,4-dimethoxycyclohexa-2,5-dien-1-one (II-1) are obtained.

### Synthesis of 4,4-dimethoxycyclohexanone (I-1) [56180-50-8]

154 g (0.958 mol, 95.7% purity) of 4,4-dimethoxycyclohexa-2,5-dien-1-one (II-1) and 10.8 g (0.083 mol) of N,N-diisopropylethylamine are dissolved in 800 ml of methyltetrahydrofuran and hydrogenated over 1.54 g of palladium 5% on activated carbon with 100 bar of hydrogen until the pressure is constant. The autoclave is cooled so that the reaction temperature does not exceed 30° C. The reaction mixture is filtered through kieselguhr. Removal of the solvent results in 153 g (91.9% purity, 92% yield) of 4,4-dimethoxycyclohexanone (I-1).

### Synthesis of 1,4,9,12-tetraoxadispiro[4.2.4.2]tetradeca-6,13-diene [35375-33-6]

1000 g (5 mol) of 3,3,6,6-tetramethoxycyclohexa-1,4-dione are suspended in 2000 ml of 1,2-ethanediol. At 5° C., 0.6 g of p-toluenesulphonic acid is added, and the suspension is stirred at 5° C. for 2 hours. The reaction is followed by gas chromatography. To complete the precipitation, the reaction mixture is cooled to 0° C. The solid is filtered off, washed with 1000 ml of cold water and dried at room temperature in vacuo. 876 g (4.46 mol, 100% purity, 89% yield) of 1,4,9,12-tetraoxa-dispiro[4.2.4.2]tetradeca-6,13-diene are obtained.

### Synthesis of 1,4-dioxaspiro[4.5]deca-6,9-dien-8-one (II-2) [35357-34-7]

813 g (4.12 mol) of 1,4,9,12-tetraoxadispiro[4.2.4.2]tetradeca-6,13-diene are stirred in a mixture of 1600 ml of tetrahydrofuran, 1600 ml of water and 32 ml of acetic acid at 64° C. for 6 hours. The reaction is followed by gas chromatography. After the monohydrolysis is complete, the tetrahydrofuran is stripped off in vacuo. The aqueous residue is extracted 2× with 1000 ml of toluene. The combined organic extracts are stirred with 20 g of K₂CO₃, dried with Na₂SO₄ and concentrated in a rotary evaporator. 570 g (3.74 mol, 91% yield) of 1,4-dioxaspiro[4.5]deca-6,9-dien-8-one (II-2) are obtained.

### Synthesis of 1,4-dioxaspiro[4.5]decan-8-one (I-2) [4746-97-8]

10 g (0.66 mol) of 1,4-dioxaspiro[4.5]deca-6,9-dien-8-one and 0.7 g (5.4 mmol) of N,N-diisopropylethylamine are dissolved in 200 ml of methyltetrahydrofuran and hydrogenated over 0.1 g of palladium 5% on activated carbon with 100 bar of hydrogen until the pressure is constant. The autoclave is cooled so that the reaction temperature does not exceed 30° C. The reaction mixture is filtered through kieselguhr. Removal of the solvent results in 9.4 g (91% yield) of 1,4-dioxaspiro[4.5]decan-8-one (I-2).

### Synthesis of 1,5,10,14-tetraoxadispiro[5.2.5.2]hexadeca-7,15-diene [77746-35-1]

50 g (0.25 mol) of 3,3,6,6-tetramethoxycyclohexa-1,4-dione are suspended in 500 ml of 1,3-propanediol. At 0° C., 50 mg of p-toluenesulphonic acid are added, and the suspension is stirred at 0° C. for 3 hours. To complete the precipitation, 300 ml of saturated NaHCO₃ solution are added. The solid is filtered off, washed with 200 ml of cold water and dried at room temperature in vacuo. 46.1 g (0.207 mol, 82.3% yield) of 1,5,10,14-tetraoxadispiro[5.2.5.2]hexadeca-7,15-diene are obtained.

### Synthesis of 1,5-dioxaspiro[5.5]undeca-7,10-dien-9-one (II-3) [67856-46-6]

95.2 g (0.425 mol) of 1,5,10,14-tetraoxadispiro[5.2.5.2]hexadeca-7,15-diene are stirred in a mixture of 340 ml of tetrahydrofuran, 170 ml of water and 5.1 ml of acetic acid at 70° C. for 7 hours. After the monohydrolysis is complete, the tetrahydrofuran is stripped off in vacuo. The aqueous residue is mixed with 100 ml of NaHCO₃ solution and extracted 2× with 300 ml of tert-butyl methyl ether. The combined organic extracts are dried with Na₂SO₄, filtered through basic alumina and concentrated in a rotary evaporator. 58.0 g (0.35 mol, 83.8% yield) of 1,5-dioxaspiro[5.5]undeca-7,10-dien-9-one are obtained.

### Synthesis of 1,5-dioxaspiro[5.5]undecan-9-one (I-3) [76626-13-6]

58 g (0.35 mol) of 1,5-dioxaspiro[5.5]undeca-7,10-dien-9-one (II-3) and 5.8 ml of triethylamine are dissolved in 21 of toluene and hydrogenated over 5.8 g of palladium 10% on activated carbon with 100 bar of hydrogen until the pressure is constant. The autoclave is cooled so that the reaction temperature does not exceed 20° C. The reaction mixture is filtered through kieselguhr, and removal of the solvent results in 51.6 g (0.3 mol, 86.7% yield) of 1,5-dioxaspiro[5.5]undecan-9-one.

1. Process for preparing compounds of the formula (I) 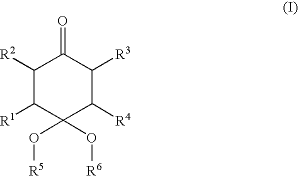
in which
R¹, R², R³, R⁴ independently of one another are hydrogen or are in each case optionally mono- or polysubstituted C₁-C₄-alkyl or cyclopropyl,
R⁵ and R⁶ independently of one another are C₁-C₈-alkyl or are cycloalkyl, or
R⁵ and R⁶ together are —CH₂—, —CH₂CH₂—, —CH₂CH₂CH₂—, —CH₂CH₂CH₂CH₂—, —CHCH₃CH₂CHCH₃CH₂—, —CH₂C(CH₃)₂CH₂—, —CH₂OCH₂—, —CH₂OCH₂CH₂—, —CH₂CH₂OCH₂—,
characterized in that
compounds of the formula (II) 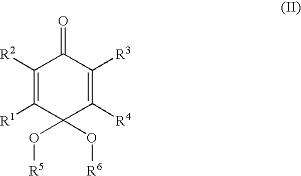
in which
R¹, R², R³, R⁴, R⁵, R⁶ have the meanings indicated above,
are hydrogenated in the presence of a suitable metal catalyst, of a suitable additive and where appropriate of a solvent.

2. Process for preparing compounds of the formula (I) according to claim 1, in which
R¹, R², R³, R⁴ independently of one another are hydrogen or are methyl, ethyl, i-propyl, t-butyl or cyclopropyl,
R⁵ and R⁶ independently of one another are methyl, ethyl, i-propyl, t-butyl or are cyclopropyl, cyclobutyl, cyclopentyl, or cyclohexyl or
R⁵ and R⁶ together are —CH₂—, —CH₂CH₂—, —CH₂CH₂CH₂—, —CH₂CH₂CH₂CH₂—, —CHCH₃CH₂CHCH₃CH₂— or —CH₂C(CH₃)₂CH₂—.

3. Process for preparing compounds of the formula (I) in which
R¹, R², R³, R⁴ independently of one another are hydrogen, methyl or ethyl,
R⁵ and R⁶ independently of one another are methyl, ethyl, i-propyl or t-butyl or
R⁵ and R⁶ together are —CH₂—, —CH₂CH₂—, —CH₂CH₂CH₂—, —CHCH₃CH₂CHCH₃CH₂— or —CH₂C(CH₃)₂CH₂—.

4. Process for preparing compounds of the formula (I) in which
R¹, R², R³, R⁴ independently of one another are hydrogen or methyl,
R⁵ and R⁶ independently of one another are methyl, or
R⁵ and R⁶ together are —CH₂CH₂— or —CH₂CH₂CH₂—.

5. Process for preparing compounds of the formula (I) in which
R¹, R², R³, R⁴ independently of one another are hydrogen,
R⁵ and R⁶ independently of one another are methyl.

6. Process for preparing compounds of the formula (I) in which
R¹, R², R³, R⁴ independently of one another are hydrogen,
R⁵ and R⁶ together are —CH₂—CH₂—.

7. Process for preparing compounds of the formula (I) in which
R¹, R², R³, R⁴ independently of one another are hydrogen,
R⁵ and R⁶ together are —CH₂CH₂CH₂—.

8. Process for preparing compounds of the formula (I) according to claims 1-7 characterized in that an organic base is employed as additive.

9. Process according to claim 8, characterized in that N,N-diisopropylethylamine is employed as additive.

10. Process according to claim 8, characterized in that triethylamine is employed as additive.

11. Process according to claim 1-10, characterized in that a palladium metal catalyst is employed.
